(19) United States
(12) Patent Application Publication
(10) Pub. No.: US 2007/0141678 A1
(43) Pub. Date: Jun. 21, 2007

(54) Processes for purifying oligosaccharides

(75) Inventors: Phillip Richard Green, Cincinnati, OH (US); Karunakaran Narasimhan, West Chester, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company

(21) Appl. No.: 11/604,453

(22) Filed: Nov. 27, 2006

Related U.S. Application Data
(60) Provisional application No. 60/752,251, filed on Dec. 19, 2005.

(51) Int. Cl. C12P 19/28; C08B 37/00

(52) U.S. Cl. 435/85; 536/18.7

(57) ABSTRACT

Processes for purifying oligosaccharides including contacting a basic oligosaccharide solution with a basic boronate resin to bind monosaccharides to the basic boronate resin and obtain a purified oligosaccharide solution. The basic boronate resin can bind about 50% of the monosaccharides and about 10% of oligosaccharides in the basic oligosaccharide solution, by weight of total carbohydrates therein.

### CROSS-REFERENCE TO RELATED APPLICATIONS

This application claims the benefit of Provisional U.S. Patent Application No. 60/752,251 filed Dec. 19, 2005.

### FIELD OF THE INVENTION

Exemplary embodiments of the present invention generally relate to processes for purifying oligosaccharides by removing monosaccharide byproducts.

### BACKGROUND OF THE INVENTION

Oligosaccharides, such as fructooligosaccharide (FOS) and galactooligosaccharide (GOS) are nutritive materials that may be incorporated into foods and beverages to help improve intestinal health. More specifically, oligosaccharides, which are also known as “prebiotics,” can aid in the improvement of intestinal health by stimulating the growth of beneficial bacteria such as Bifidobacteria.

FOS and GOS may be produced enzymatically from sucrose or lactose, respectively. In addition, FOS and GOS may release the monosaccharides fructose and glucose, or galactose and glucose, respectively, as byproducts. Because the intestinal health benefits are related to the oligosaccharides, rather than the monosaccharide byproducts, a highly purified oligosaccharide is desirable. To achieve such highly purified oligosaccharides, the monosaccharide byproducts must be removed. However, chemical removal of the monosaccharide byproducts can be costly and complex since the monosaccharide byproducts sought to be removed have similar properties to the oligosaccharides (e.g. the oligosaccharides and monosaccharides are both carbohydrates that can react in a similar manner).

Thus, there remains a need for cost effective processes by which to remove monosaccharide byproducts from oligosaccharide nutrients, thereby purifying the oligosaccharides.

### SUMMARY OF THE INVENTION

Exemplary embodiments of the present invention generally relate to processes for purifying oligosaccharides comprising contacting a basic oligosaccharide solution with a basic boronate resin to bind monosaccharides to the basic boronate resin and obtain a purified oligosaccharide solution, wherein the basic boronate resin binds at least about 50% of the monosaccharides and less than about 10% of oligosaccharides in the basic oligosaccharide solution, by weight of total carbohydrates therein.

In addition, exemplary embodiments of the present invention may relate to processes for purifying oligosaccharides comprising providing a crude oligosaccharide solution and a boronate resin, adjusting pH of the crude oligosaccharide solution to obtain a basic oligosaccharide solution, adjusting pH of the boronate resin to about the pH of the basic oligosaccharide solution to obtain a basic boronate resin, and contacting the basic oligosaccharide solution with the basic boronate resin to bind monosaccharides to the basic boronate resin and obtain a purified oligosaccharide solution wherein the basic boronate resin binds at least about 50% of the monosaccharides and less than about 10% of oligosaccharides in the basic oligosaccharide solution, by weight of total carbohydrates therein.

In addition, exemplary embodiments of the present invention may relate to processes for purifying oligosaccharides comprising contacting a basic oligosaccharide solution having a pH of from about 8 to about 10 with a basic boronate resin having a pH of from about 8 to about 10 to bind monosaccharides to the basic boronate resin and obtain a purified oligosaccharide solution, wherein the basic boronate resin binds from about 90% to about 99.9% of the monosaccharides and from about 0.1% to about 5% of oligosaccharides in the basic oligosaccharide solution, by weight of total carbohydrates therein.

### DETAILED DESCRIPTION OF THE INVENTION

Exemplary embodiments of the present invention generally relate to processes for purifying oligosaccharides by removing monosaccharide byproducts.

### A. Definitions

As used herein, the term “basic boronate resin” refers to the condition of a boronate resin after the pH is adjusted to about the same pH as the basic oligosaccharide solution. The pH may be adjusted by adding a base to the boronate resin. While any base may be suitable for use herein, in one embodiment, the base may be selected from the group consisting of sodium hydroxide, calcium hydroxide, ammonium hydroxide, potassium hydroxide, alkaline water, ionized water with OH⁻ and combinations thereof, to the boronate resin. In an exemplary embodiment, the basic boronate resin may comprise a pH of at least about 7.5, and in another exemplary embodiment, a pH of from about 8 to about 10.

As used herein, the term “basic oligosaccharide solution” refers to a crude oligosaccharide solution after the pH of the crude oligosaccharide solution has been adjusted using a base selected from the group consisting of sodium hydroxide, calcium hydroxide, ammonium hydroxide, potassium hydroxide, alkaline water, ionized water with OH⁻ and combinations thereof. In an exemplary embodiment, the basic oligosaccharide solution may comprise a pH of at least about 7.5, and in another exemplary embodiment, from about 8 to about 10.

As used herein, “boronate resin” means a fixed bed or column of insoluble material with which boronate has been reacted such that the boronate becomes immobilized on the insoluble material such that it is no longer free to intermix with either the basic oligosaccharide solution or the resulting purified oligosaccharide solution. As used herein, “boronate” means any boronate derivative capable of being immobilized on a chemically reactive resin or substratum. In one embodiment, suitable boronate derivatives for use herein may be selected from the group consisting of ortho-aminophenyl boronate, meta-aminophenyl boronate, para-aminophenyl boronate and combinations thereof.

As used herein, the term “comprising” means various components can be cojointly employed in the methods and articles of this invention. Accordingly, the terms “consisting essentially of” and “consisting of” are embodied in the term comprising.

As used herein, the term “crude oligosaccharide solution” means a solution comprising oligosaccharides, monosaccharides, salts (such as, calcium acetate) and water.

As used herein, the term “monosaccharide(s)” means a sugar selected from the group consisting of glucose, fructose, galactose, xylose and combinations thereof.

As used herein, the term “oligosaccharide(s)” means a compound comprising at least two or more sugars, selected from the group consisting of glucose, fructose, galactose, xylose and combinations thereof. In one embodiment, the oligosaccharide may be selected from the group consisting of fructooligosaccharide, galactooligosaccharide, lactosucrose, isomaltulose, glycosyl sucrose, isomaltooligosaccharide, gentioligosaccharide, xylooligosaccharide and combinations thereof. As used herein, the term “oligosaccharides” includes disaccharides.

As used herein, the term “purified oligosaccharide solution” refers to solution that results from removing at least about 50% of the monosaccharides present in a crude oligosaccharide solution. The purified oligosaccharide solution may comprise oligosaccharides, monosaccharides, salts and water. The purified oligosaccharide solution may be dried to produce a “purified oligosaccharide composition” that may comprise at least about 75% oligosaccharides, and in one embodiment at least about 80% oligosaccharide, and in yet another embodiment at least about 90% oligosaccharide, by weight of the total carbohydrates.

As used herein, the term “total carbohydrates” refers to a portion of any of the crude and/or purified oligosaccharide solution or purified oligosaccharide composition that comprises carbohydrates, including, but not limited to, monosaccharides and oligosaccharides. For example, in one exemplary embodiment, the purified oligosaccharide composition may comprise at least about 75% oligosaccharide by weight of the total carbohydrates present in the purified solution. This means that about 25%, by weight of the total carbohydrates, may comprise other carbohydrates, such as monosaccharides.

### B. Processes

Exemplary embodiments of the present invention generally relate to processes for purifying oligosaccharides comprising contacting a basic oligosaccharide solution with a basic boronate resin to bind monosaccharides to the basic boronate resin and obtain a purified oligosaccharide solution, wherein the basic boronate resin binds at least about 50% of the monosaccharides and less than about 10% of oligosaccharides in the basic oligosaccharide solution, by weight of total carbohydrates therein.

Embodiments of the processes herein may initially comprise providing a crude oligosaccharide solution. As used herein, “oligosaccharide(s)” means a compound comprising at least two or more sugars selected from the group consisting of glucose, fructose, galactose, xylose and combinations thereof. In one embodiment, the oligosaccharide may be selected from the group consisting of fructooligosaccharide, galactooligosaccharide, lactosucrose, isomaltulose, glycosyl sucrose, isomaltooligosaccharide, gentioligosaccharide, xylooligosaccharide and combinations thereof. A “crude oligosaccharide solution” herein may comprise oligosaccharides, monosaccharides, salts (such as calcium acetate) and water. Because any amount of water may be present in the crude oligosaccharide solution, it will be understood by those skilled in the art that the concentration of oligosaccharides in the crude oligosaccharide solution can vary.

Prior to adjusting the pH of the crude oligosaccharide solution (discussed later herein) the crude oligosaccharide solution may optionally be treated with a catalytic enzyme in order to aid in hydrolyzing disaccharides present in the solution. Catalytic enzymes acceptable for use herein may include, but are not limited to, α-glucosidase and β-galactosidase. If the crude oligosaccharide solution is treated with α-glucosidase, any sucrose present in the crude oligosaccharide solution can be hydrolyzed to produce glucose and fructose. If the crude oligosaccharide solution is treated with β-galactosidase, any lactose present in the crude oligosaccharide solution can be hydrolyzed to produce glucose and galactose.

Embodiments of the processes herein may also initially comprise providing a boronate resin. “Boronate” means any boronate derivative capable of being immobilized on a chemically reactive resin or substratum. In one embodiment, suitable boronate derivatives for use herein may be selected from the group consisting of ortho-aminophenyl boronate, meta-aminophenyl boronate, para-aminophenyl boronate and combinations thereof. As used herein, “boronate resin” refers to a fixed bed or column of insoluble material (e.g. resin) with which boronate has been reacted such that the boronate becomes immobilized on the insoluble material such that it is no longer free to intermix with either the basic oligosaccharide solution or the resulting purified oligosaccharide solution. Suitable insoluble materials acceptable for use herein include, but are not limited to, minerals such as silica, polymers such as cellulose, copolymers such as N,N′-methylene-bis-(methacrylamide) and combinations thereof. Those skilled in the art will understand how to properly select an insoluble material for use herein. As discussed later herein, by immobilizing the boronate on a resin, the boronate can be prevented from intermixing with the purified oligosaccharide solution, thereby further enhancing the purity thereof. This can improve the cost efficiency of the processes since further purification of the oligosaccharide is not necessarily needed before use. Moreover, immobilizing the boronate can allow for the regeneration of the boronate which, as described later herein, can also increase the cost efficiency of the process.

After obtaining, and optionally treating, the crude oligosaccharide solution, the pH of both the crude oligosaccharide solution and the boronate resin may be adjusted to obtain a basic oligosaccharide solution and a basic boronate resin. Ordinarily, both the crude oligosaccharide solution and the boronate resin may have an acidic pH. However, through pH manipulation, the crude oligosaccharide solution and the boronate resin can be made basic. For example, in one exemplary embodiment, the crude oligosaccharide solution may be adjusted to yield a basic oligosaccharide solution comprising a pH of at least about 7.5, and in another exemplary embodiment, from about 8 to about 10. The pH of the boronate resin may also be adjusted to approximately the same pH as the basic oligosaccharide solution. Acceptable bases for adjusting the pH of both the crude oligosaccharide solution and the boronate resin may include, for example, sodium hydroxide, calcium hydroxide, ammonium hydroxide, potassium hydroxide, alkaline water, ionized water with OH⁻ and combinations thereof. However, it should be understood that any suitable base is acceptable for use herein. Those skilled in the art will understand how to select the proper concentration of base needed to adjust the crude oligosaccharide solution and boronate resin to the desired pH.

It has been surprisingly discovered that employing basic conditions, as set forth herein, can enhance purification of the oligosaccharides. Without intending to be limited by theory, it is believed that under basic conditions, steric hindrance can limit the binding of oligosaccharides to the boronate. This can hold true until the pH is increased to very basic conditions (i.e. pH of greater than about 10). As discussed below, it is believed that the basic boronate resin can preferentially bind the monosaccharide byproducts, rather than the desired oligosaccharides, thus yielding a more purified oligosaccharide. As previously discussed, this can improve the cost efficiency of the process as it can reduce or eliminate the need for further purification of the oligosaccharide prior to use.

Having adjusted pH, the basic oligosaccharide solution may then be contacted with the basic boronate resin. Upon contacting the basic oligosaccharide solution with the basic boronate resin, the basic boronate resin may bind at least about 50% monosaccharides, and in one embodiment at least about 75% monosaccharides, and in yet another embodiment from about 90% to about 99.9% monosaccharides, and in still another embodiment from about 95% to about 99.9% monosaccharides present in the basic oligosaccharide solution, by weight of total carbohydrates therein. It is believed that as the pH of the system (i.e. the resin and the oligosaccharide solution) approaches about 10, the ability of the boronate resin to preferentially bind monosaccharides in the basic oligosaccharide solution, rather than oligosaccharides, can be improved. As used herein, the term “total carbohydrates” refers to a portion of any of the crude and/or purified oligosaccharide solutions or purified oligosaccharide composition that comprises carbohydrates, including, but not limited to, monosaccharides and oligosaccharides. In contrast to the monosaccharides, the basic boronate resin may bind less than about 10% oligosaccharides, and in one embodiment from about 0.1% to about 5% oligosaccharides present in the basic oligosaccharide solution, by weight of the total carbohydrates.

The resulting purified oligosaccharide solution may then be dried to produce a purified oligosaccharide composition. Drying may be carried out using any conventional drying method known to those skilled in the art. For example, drying may be accomplished by a spray dryer, rotary drum dryer, tray dryer or screw dryer.

In one embodiment, the purified oligosaccharide composition may comprise at least about 75%, and in one embodiment at least about 80%, and in yet another embodiment at least about 90%, and in still another embodiment from about 95% to about 99.9% of oligosaccharides, by weight of total carbohydrates therein.

Additionally, and as previously mentioned, to further improve the cost effectiveness of the exemplary processes herein, it may be desirable to regenerate the basic boronate resin comprising the bound monosaccharides. Such regeneration may be accomplished by a variety of means including, but not limited to, contacting an acid, or acid solution, with the basic boronate resin to make the pH of the boronate resin acidic. This allows the bound monosaccharides to separate from the boronate resin and the boronate resin to be reused. In one embodiment, an acid may be added to the basic boronate resin until the pH of the boronate resin is from about 3.5 to about 6. Suitable acids for use herein include, but are not limited to, mineral acids (e.g. sulfuric or hydrochloric), organic acids (e.g. acetic), ionized water with H⁺ and combinations thereof.

It will also be understood by those skilled in the art that the processes described herein may be carried out using conventional batch or continuous processes known to those skilled in the art. Batch processes acceptable for use herein may include, but are not limited to, column chromatography or batch packed beds with sequenced feed, product and regeneration streams. Similarly, continuous processes acceptable for use herein may include, but are not limited to, processes involving the use of multiple chromatography beds, or packed beds with simultaneous feed, product and regeneration streams, accomplished using a ‘Simulated Moving Bed’ concept.

### EXAMPLES

### Example 1

Affigel® Boronate resin is hydrated in water and packed into an Amersham Pharmacia C16/20 column. To adjust the pH, the boronate resin is washed in 50 mM ammonium hydroxide until the pH of the eluate is about the same as the ammonium hydroxide feed. The boronate resin is then washed with 50 mM acetic acid until the pH of the eluate is about the same as the acetic acid feed. The resin is then treated with 100 mM ammonium acetate and a pH of about 10.0 is obtained, thereby providing a basic boronate resin.

Crude galactooligosaccharide (GOS) (23% GOS, 45% lactose, 22% glucose and 10% galactose) is dissolved in about 50 mL of 100 mM ammonium acetate to adjust the pH of the solution and produce a basic GOS solution having a pH of about 10. The basic GOS solution is applied to the column containing the basic boronate resin and washed with about 50 mL of 100 mM ammonium acetate, pH about 10.0, and the unbound oligosaccharides are collected to yield a purified GOS solution.

To regenerate the column, about 100 mL of 100 mM acetic acid is applied to the column to bring the column to a pH of about 3.5 and the acid eluate is collected. The purified GOS solution and the acetic acid eluate are analyzed by High Performance Liquid Chromatography over a 6.5×300 mm Water's Sugar-Pak 1 column using a 0.001 mM calcium chloride-EDTA mobile phase at about 80° C., 0.6 mL per minute and detected using a Water's 2414 Refractive Index Detector. The purified GOS solution contains about 38% GOS and about 62% lactose, by weight of total carbohydrates therein. Thus, the monosaccharides glucose and galactose have been successfully removed therefrom. The acetic acid eluate contains the monosaccharides glucose and galactose, as well as some residual GOS that had bound to the basic boronate resin.

### Example 2

Affigel® Boronate resin is suspended in water and sodium hydroxide is added until the pH is about 11.5 and a basic boronate resin is obtained. The basic boronate resin is then washed two times in 5 mM sodium hydroxide. Crude fructooligosaccharide (FOS) is dissolved into about 45 mL of 5 mM sodium hydroxide and into about 45 mL of 5 mM calcium hydroxide to provide two basic FOS solutions. About 5 mL of the basic boronate resin is added to each solution and mixed for about 1 hour. The resin is removed from each solution by centrifugation and washed two times with about 50 mL of 5 mM sodium hydroxide or calcium hydroxide, respectively. The two sodium hydroxide washes are combined to provide a first purified FOS solution and the two calcium hydroxide washes are combined to provide a second purified FOS solution. The resin is then suspended in about 50 mL of 100 mM acetic acid and mixed for about 10 minutes. The resin is then removed via centrifugation and the acetic acid solution is collected. Both purified FOS solutions, as well as the acetic acid solution are analyzed as in Example 1. FOS and sucrose are found in the sodium and calcium hydroxide solutions, and glucose and fructose are found in the acetic acid solution. About 50 mL of the purified FOS solution obtained using calcium hydroxide is further treated with 16 μL concentrated sulfuric acid or 10 μL phosphoric acid, and the precipitated calcium salt is removed by centrifugation or filtration. The FOS and sucrose are still found in solution.

### Example 3

The pH of a crude FOS solution is adjusted to about 9.5 using 100 mM ammonium acetate to obtain a basic FOS solution. A column of Affigel® Boronate resin is washed in 50 mM ammonium hydroxide, followed by 50 mM acetic acid and the top 5% of the boronate resin in the column is treated with an ammonium acetate buffer to obtain a basic boronate resin having a pH of about 9.5. The basic FOS solution is then added to the basic boronate column at a concentration such that the ammonium acetate present in the basic FOS solution is sufficient to adjust the pH of the boronate resin ahead of the boronate saturated with bound glucose and fructose. Basic FOS solution is continually added to the column until the basic boronate resin is saturated with glucose and fructose, as detected by increasing levels of glucose and fructose coming off of the column. The basic boronate resin having bound monosaccharides is then regenerated by passing an acid through the column to release the bound glucose and fructose.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A process for purifying oligosaccharides comprising:
contacting a basic oligosaccharide solution with a basic boronate resin to bind monosaccharides to the basic boronate resin and obtain a purified oligosaccharide solution,
wherein the basic boronate resin binds at least about 50% of the monosaccharides and less than about 10% of oligosaccharides in the basic oligosaccharide solution, by weight of total carbohydrates therein.

2. The process of claim 1 wherein the boronate resin comprises a boronate derivative selected from the group consisting of ortho-aminophenyl boronate, meta-aminophenyl boronate, para-aminophenyl boronate and combinations thereof.

3. The process of claim 1 wherein the oligosaccharides comprise two or more sugars selected from the group consisting of glucose, fructose, galactose, xylose and combinations thereof.

4. The process of claim 1 wherein the oligosaccharides are selected from the group consisting of fructooligosaccharide, galactooligosaccharide, lactosucrose, isomaltulose, glycosyl sucrose, isomaltooligosaccharide, gentioligosaccharide, xylooligosaccharide and combinations thereof.

5. The process of claim 1 wherein the basic oligosaccharide solution and the basic boronate resin each comprise a pH of at least about 7.5.

6. The process of claim 1 wherein the basic boronate resin binds at least about 75% of the monosaccharides and less than about 10% of the oligosaccharides in the basic oligosaccharide solution, by weight of total carbohydrates.

7. The process of claim 1 wherein the basic boronate resin binds from about 90% to about 99.9% of the monosaccharides and from about 0.1% to about 5% of the oligosaccharides in the basic oligosaccharide solution, by weight of total carbohydrates.

8. The process of claim 1 comprising drying the purified oligosaccharide solution to obtain a purified oligosaccharide composition.

9. The process of claim 1 comprising:
regenerating the basic boronate resin comprising bound monosaccharides by contacting an acid with the basic boronate resin to obtain an acidic boronate resin; and
separating the bound monosaccharides from the acidic boronate resin.

10. The process of claim 9 wherein the acid is selected from the group consisting of mineral acids, organic acids, ionized water with H⁺ and combinations thereof.

11. The process of claim 8 wherein the purified oligosaccharide composition comprises at least about 75% oligosaccharides by weight of total carbohydrates.

12. A process for purifying oligosaccharides comprising:
providing a crude oligosaccharide solution and a boronate resin;
adjusting pH of the crude oligosaccharide solution to obtain a basic oligosaccharide solution;
adjusting pH of the boronate resin to about the pH of the basic oligosaccharide solution to obtain a basic boronate resin; and
contacting the basic oligosaccharide solution with the basic boronate resin to bind monosaccharides to the basic boronate resin and obtain a purified oligosaccharide solution
wherein the basic boronate resin binds at least about 50% of the monosaccharides and less than about 10% of oligosaccharides in the basic oligosaccharide solution, by weight of total carbohydrates therein.

13. The process of claim 16 comprising treating the crude oligosaccharide solution with a catalytic enzyme selected from the group consisting of α-glucosidase, β-galactosidase and combinations thereof.

14. The process of claim 16 comprising adjusting the pH of the crude oligosaccharide solution to at least about 7.5 by adding a base selected from the group consisting of sodium hydroxide, calcium hydroxide, ammonium hydroxide, potassium hydroxide, alkaline water, ionized water with OH⁻ and combinations thereof.

15. The process of claim 16 wherein the boronate resin comprises a boronate derivative selected from the group consisting of ortho-aminophenyl boronate, meta-aminophenyl boronate, para-aminophenyl boronate and combinations thereof.

16. The process of claim 16 wherein the oligosaccharides comprise two or more sugars selected from the group consisting of glucose, fructose, galactose, xylose and combinations thereof.

17. The process of claim 16 wherein the basic boronate resin binds at least about 75% of the monosaccharides and less than about 10% of the oligosaccharides in the basic oligosaccharide solution, by weight of total carbohydrates.

18. The process of claim 16 comprising adjusting the pH of the boronate resin to at least about 7.5 using a base selected from the group consisting of sodium hydroxide, calcium hydroxide, ammonium hydroxide, potassium hydroxide, alkaline water, ionized water with OH⁻ and combinations thereof.

19. The process of claim 16 wherein the basic boronate resin binds from about 90% to about 99.9% of the monosaccharides and from about 0.1% to about 5% of the oligosaccharides in the basic oligosaccharide solution, by weight of total carbohydrates.

20. A process for purifying oligosaccharides comprising:
contacting a basic oligosaccharide solution having a pH of from about 8 to about 10 with a basic boronate resin having a pH of from about 8 to about 10 to bind monosaccharides to the basic boronate resin and obtain a purified oligosaccharide solution,
wherein the basic boronate resin binds from about 90% to about 99.9% of the monosaccharides and from about 0.1% to about 5% of oligosaccharides in the basic oligosaccharide solution, by weight of total carbohydrates therein.
